(19) United States
(12) Patent Application Publication
(10) Pub. No.: US 2023/0372644 A1
(43) Pub. Date: Nov. 23, 2023

(54) Terpene Inhalant

(71) Applicants: Anthony Contino, Bushkill, PA (US); Dacia Boehning, Jim Thorpe, PA (US)

(72) Inventors: Anthony Contino, Bushkill, PA (US); Dacia Boehning, Jim Thorpe, PA (US)

(21) Appl. No.: 17/750,695

(22) Filed: May 23, 2022

(51) Int. Cl. A61M 15/00; A61K 9/00

(52) U.S. Cl. CPC A61M 15/0031; A61K 9/007; A61M 15/0086

(57) ABSTRACT

An inhalant includes an absorbent pad containing a terpene formulation that can calm, energize, open up airways, increase clarity and enhance cognitive function on its own or in conjunction with the cannabis experience including but not limited to converting the THC jitters to a more pleasant mellow experience. The absorbent pad within a vessel to keep formulation and salts stored, A salt adjacent to the absorbent pad within the vessel to emits negative ions so when inhaling through the olfactory passageway it enters the bloodstream and increases serotonin thus giving a sense of well-being.

### BACKGROUND

The present disclosure relates to an inhalant, and more specifically to a compact vessel containing a terpene-based scent.

Terpenes are naturally occurring chemical compounds found in plants and some animals. They're responsible for the aromas, flavors, and even colors associated with various types of vegetation. In terms of cannabis, terpenes are what make certain strains smell or taste different from others. While cannabinoids provide a major element of the medicinal or psychoactive effect of Cannabis, the many terpenes in the Cannabis plant also contribute significantly to the plant's properties. Most terpenes have medical and therapeutic properties. They can be anti-microbial, anti-viral, and anti-inflammatory. They can also have a profound effect on a person's mood and sense of well-being.

### SUMMARY

An inhalant according to one disclosed non-limiting embodiment of the present disclosure includes an absorbent pad containing a terpene formulation, the absorbent pad within a vessel, A salt adjacent to the absorbent pad within the vessel.

The foregoing features and elements may be combined in various combinations without exclusivity, unless expressly indicated otherwise. These features and elements as well as the operation thereof will become more apparent in light of the following description and the accompanying drawings. It should be appreciated that however the following description and drawings are intended to be exemplary in nature and non-limiting.

### BRIEF DESCRIPTION OF THE DRAWINGS

Various features will become apparent to those skilled in the art from the following detailed description of the disclosed non-limiting embodiment. The drawings that accompany the detailed description can be briefly described as follows:

FIG. 1 is an exploded view of a terpene inhalant according to one disclosed non-limiting embodiment.

### DETAILED DESCRIPTION

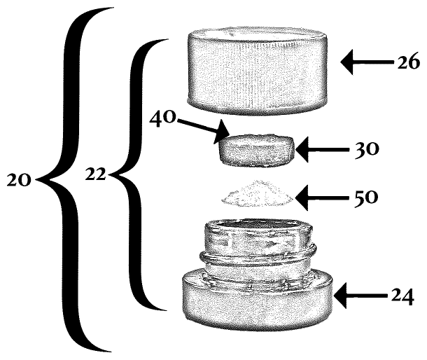

FIG. 1 schematically illustrates a terpene inhalant 20. The inhalant 20 includes a vessel 22, an absorbent pad 30 containing a terpene Terpene Formulation 40 and a layer of salt 50. The vessel 22 may be including but not limited to a round glass jar 24 and a screw-on top 26. In one example the vessel 22 is a 5 ml round glass jar or a metal, plastic, plunger or single use crushable ampule for portability.

The absorbent pad 30 may be a felt pad that is including but not limiting to, in one example, ¾ inches in diameter and ⅜ inch thick including natural fiber, synthetic fiber and combined fibers. The absorbent pad 30 contains a terpene 40 of various formulas. The terpenes may be mood specific with a core terpene formula. The formula may be all natural, vegan, and cruelty free and may include, for example, monoterpenes, sesquiterpenes, diterpenes, sesterpenes, triterpenes, and including but not limited to full plant extracts.

Below the absorbent pad 30, the layer of salt 50 may be a mineral salt such as pink Himalayan salts which are anti-bacterial, anti-inflammatory and mucus thinning to open airways. The salt 50 emits negative ions so when inhaling through the olfactory passageway it enters the bloodstream and increases serotonin thus giving a sense of well-being. Elements of construction or formulation may include but are not limited to granular, rock, powdered, and saturated solution of salts.

The terpene inhalant 20 facilitates mood adjustment. Depending on the formulation, terpenes can calm, energize, open up airways, increase clarity and enhance cognitive function on its own or in conjunction with the cannabis experience including but not limited to converting the THC jitters to a more pleasant mellow experience.

Although the different non-limiting embodiments have specific illustrated components, the embodiments of this invention are not limited to those particular combinations. It is possible to use some of the components or features from any of the non-limiting embodiments in combination with features or components from any of the other non-limiting embodiments. Including but not limited to: example a: a mood specific terpene inhalant formula on a felt pad, with salt, inside a 5 ml disposable glass dram or example b: a terpene inhalant on individually wrapped, salt saturated, felt pad refills that insert into a portable, reusable plunger ‘necklace’.

The foregoing description is exemplary rather than defined by the limitations within. Various non-limiting embodiments are disclosed herein, however, one of ordinary skill in the art would recognize that various modifications and variations in light of the above teachings will fall within the scope of the appended claims. It is therefore to be appreciated that within the scope of the appended claims, the disclosure may be practiced other than as specifically described. For that reason the appended claims should be studied to determine true scope and content.

What is claimed is:

1. An inhalant, comprising:
a vessel;
an absorbent pad containing a terpene formulation, the absorbent pad within the vessel; and
a salt adjacent to the absorbent pad within the vessel.

2. The inhalant as recited in claim 1, wherein the vessel comprises a jar and a screw-on top.

3. The inhalant as recited in claim 2, wherein the vessel is a 5 ml round glass jar.

4. The inhalant as recited in claim 1, wherein the salt comprises a mineral salt.

5. The inhalant as recited in claim 1, wherein the absorbent pad is a felt pad.

6. The inhalant as recited in claim 5, wherein the felt pad is ¾ inches in diameter and ⅜ inch thick.

7. The inhalant as recited in claim 1, wherein the salt is between the bottom of the vessel and the absorbent pad.

8. The inhalant as recited in claim 1, wherein the vessel is a single use crushable ampule

9. An inhalant, comprising:
an absorbent pad containing a terpene formulation, the absorbent pad saturated with a salt.
